(19) United States
(12) Patent Application Publication
(10) Pub. No.: US 2008/0267874 A1
(43) Pub. Date: Oct. 30, 2008

(54) Targeted Neuronal And Glial Human Embryonic Stem Cell Line

(75) Inventor: Xianmin Zeng, Novato, CA (US)

Correspondence Address: MEDLEN & CARROLL, LLP, 101 HOWARD STREET, SUITE 350, SAN FRANCISCO, CA, 94105, US

(73) Assignee: The Buck Institute for Age Research

(21) Appl. No.: 12/056,823

(22) Filed: Mar. 27, 2008

Related U.S. Application Data
(60) Provisional application No. 60/920,457, filed on Mar. 28, 2007.

(51) Int. Cl. A61K 49/00; C12N 5/06; A01K 67/027; A61P 43/00; C12N 5/08; C12Q 1/68; C40B 30/06

(52) U.S. Cl. 424/9.1; 435/366; 800/13; 435/6; 506/10

(57) ABSTRACT

The present invention is related to human stem cells lines comprising a targeted gene construct, and in particular to human embryonic stem cells lines (hESC) comprising a reporter gene inserted into the Olig2 locus via homologous recombination. The hESC line remains pluripotent and maintains a normal karyotype, and allows for visualization of Olig2 expression by fluorescence microscopy and sorting by FACS. Since Olig2 is important is the development of motor neurons and oligodendrocytes, the present invention provides a means to study differentiation of stem cells into motor neurons and oligodendrocytes, as well as the study of intrinsic and extrinsic factors that affect such differentiation. The hESCs of the present invention also provide a means to study and determine optimal factors and conditions for cell differentiation.

### FIELD OF THE INVENTION

The present invention is related to human stem cell lines comprising a targeted gene construct, and in particular to human embryonic stem cell lines comprising a reporter gene inserted into the Olig2 locus via homologous recombination.

### BACKGROUND OF THE INVENTION

Human stem cells have the potential to serve as an experimental model to study aspects of human development that are not accessible in vivo. Human stem cells also have the potential to be useful in the study of molecular and cellular mechanisms that are responsible for the onset and progression of disease. Menedez et al., Current Gene Ther. 5:375-85 (2005).

Gene targeting, which is the homologous recombination of DNA sequences residing in the chromosome with newly introduced DNA sequences, provides a means for specifically altering the mammalian genome. Thomas and Capecchi, Cell 51:503-512 (1987). Gene targeting has been applied to mouse embryonic stem cell (mESC) lines to study neuronal differentiation. Xian et al., Stem Cells 21:41-49 (2003) describe the creation of mESCs (RW4 ES) that contain a GFP knock-in to the Olig2 locus. Xian and Gottlieb, Glia 47:88-101 (2004) reports the use of the GFP-Olig2 knock-in mESCs (as described in Xian 2003). Xian et al., Biochem. Biophys. Res. Comm. 327:155-162 (2005) describe the use of the GFP-Olig2 knock-in mice (Xian, 2003) to develop culture conditions with RA and Shh for rapid and efficient differentiation of ES cells along neural pathway for HTS potential.

However, gene targeting in human stem cells has proven difficult. Zwaka and Thomson, Nature Biotech. 21:319-321 (2003) report that high, stable transfection efficiencies in human embryonic stem cells (hESCs) have been difficult to achieve, and that electroporation protocols established for mESCs work poorly in hESCs. Yates and Daley, Gene Ther. 13(20):1431-9)(2006) report that HR (homologous recombination) has not been as extensively employed in hESCs (compared to mESCs), and that to date only three HR-targeted hESC lines (2 HPRT lines and one Oct4 line) exist. According to the authors, this is partly due to the major difficulties encountered in transfecting DNA into hESCs. Moreover, to date, genes that are not expressed in undifferentiated human stem cells have not been targeted.

Accordingly, what is needed in the art are human stem cells lines comprising targeted mutations in genes that expressed in undifferentiated human stem cells.

### SUMMARY OF THE INVENTION

The present invention is related to human stem cell lines comprising a targeted gene construct, and in particular to human embryonic stem cell lines comprising a reporter gene inserted into the Olig2 locus via homologous recombination. Accordingly, in some embodiments, the present invention provides a human stem cell comprising a reporter gene inserted into a gene that is not expressed in said human stem cell prior to differentiation. In some embodiments, the human stem cell is an embryonic stem cell. In some embodiments, the reporter gene is inserted into olig2 locus. In some embodiments, the reporter gene encodes a fluorescent protein. In some embodiments, the fluorescent protein is selected from the group consisting of green fluorescent protein, red fluorescent protein, yellow fluorescent protein. In further embodiments, the present invention provides an isolated stem cell line comprising the foregoing human stem cells. In further embodiments, the present invention provides an in vitro cell culture comprising the foregoing human stem cells. In some embodiments, the present invention provides an organism comprising the foregoing human stem cells. In still further embodiments, the present invention provides a tissue comprising the foregoing human stem cells.

In some embodiments, the present invention provides methods of screening compounds comprising: a) providing human stem cells comprising a reporter gene inserted into a gene that is not expressed in said human stem cells prior to differentiation; b) contacting said human stem cells with said at least one compound; c) culturing said human stem cells; and d) assaying said human stem cells for expression of said reporter gene. In some embodiments, the human stem cells comprise a reporter gene inserted into the Olig2 locus. In some embodiments, the assaying is performed by a method selected from the group consisting of fluorescence microscopy and fluorescence activated cell sorting. In some embodiments, the culturing comprises culturing said human stem cells under conditions such that said human stem cell is allowed to differentiate. In some embodiments, a plurality of cells are provided and said contacting step comprises contacting said plurality of cells with a library of compounds in a high throughput assay. In some embodiments, the compound is selected from the group consisting of proteins, peptides, polypeptides and small organic compounds. In some embodiments, the human stem cells are embryonic stem cells. In some embodiments, the methods further comprise the step of selecting a compound. In some embodiments, the methods further comprise the step of testing said compound in a human animal. In some embodiments, the methods further comprise the step of packaging said compound for use in animals.

In some embodiments, the present invention further provides method of screening for neurotoxins comprising providing: a) providing human stem cells comprising a reporter gene inserted into the Olig2 locus; b) contacting said human stem cells with said at least one compound suspected of being a neurotoxin; and c) assaying said human stem cells for expression of said reporter gene. In some embodiments, the assaying is performed by a method selected from the group consisting of fluorescence microscopy and fluorescence activated cell sorting. In some embodiments, the human stem cells are embryonic stem cells.

### DESCRIPTION OF THE FIGURES

FIG. 1 provides the sequence for human Olig2.

FIG. 2 provides the sequence of an Olig2-reporter gene (green fluorescent protein) construct of the present invention.

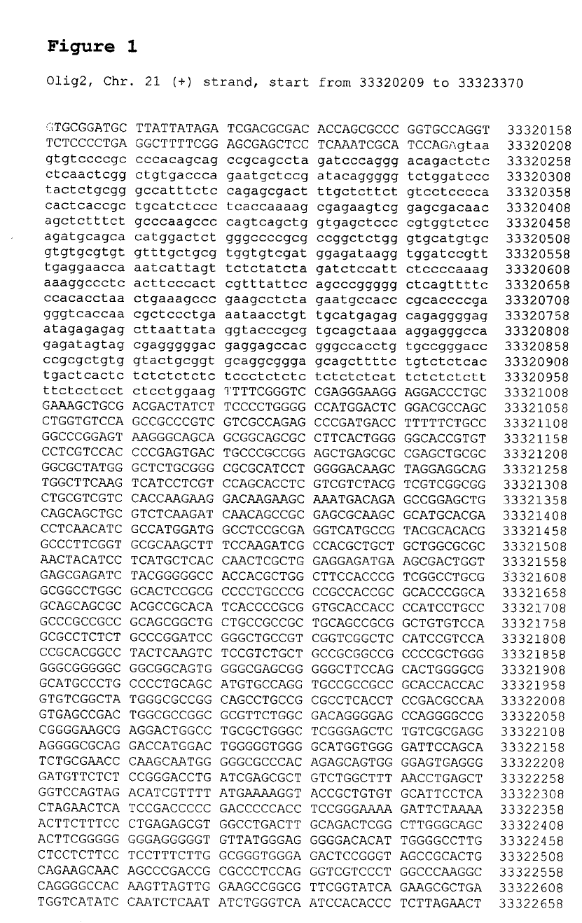

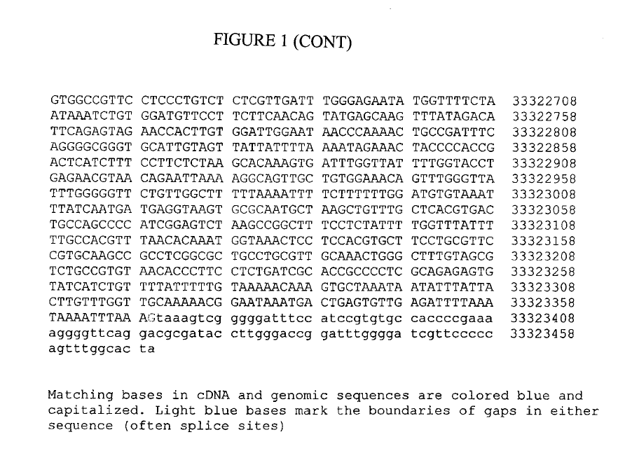

### DEFINITIONS

As used herein, the term “stem cells” means cells that are totipotent or pluripotent or multipotent and are capable of differentiating into one or more different cell types.

As used herein, the term “embryonic stem cells” means stem cells derived from an embryo of about 7 days after fertilization.

As used herein, the term “adult stem cells” means stem cells derived from an organism after birth.

As used herein, the term “mesodermal cell line” means a cell line displaying characteristics associated with mesodermal cells.

As used herein, the term “endodermal cell line” means a cell line displaying characteristics normally associated with endodermal cells.

As used herein, the term “neural cell line” means a cell line displaying characteristics normally associated with neural cell lines. Examples of such characteristics include, but are not limited to, expression of GFAP, neuron-specific enolase, Neu-N, neurofilament-N, or tau.

As used herein, the term “totipotent” means the ability of a cell to differentiate into any type of cell in a differentiated organism, as well as cell of extra embryonic materials such as placenta.

As used herein, the term “pluripotent” refers to a cell line capable of differentiating into any differentiated cell types.

As used herein, the term “multipotent” refers to a cell line capable of differentiating into at least two differentiated cell types.

As used herein, the term “differentiation” as used with respect to cells in a differentiating cell system refers to the process by which cells differentiate from one cell type (e.g., a multipotent, totipotent or pluripotent differentiable cell) to another cell type such as a target differentiated cell.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the terms “reporter gene” or “reporter construct” refer to genetic constructs comprising a nucleic acid encoding a protein that is easily detectable or easily assayable, such as a colored protein, fluorescent protein or enzyme such as beta-galactosidase.

As used herein, the term “gene targeting” refers the integration of exogenous DNA into the genome of a cell at sites where its expression can be suitably controlled. This integration occurs as a result of homologous recombination.

A “knock-in” approach as used herein refers to the procedure of inserting a desired nucleic acid sequence, such as a sequence encoding a reporter gene, into a specific locus in a host cell via homologous recombination.

As used herein, the term “genome” refers to the genetic material (e.g., chomosomes) of an organism.

The term “nucleotide sequence of interest” refers to any nucleotide sequence (e.g., RNA or DNA), the manipulation of which may be deemed desirable for any reason (e.g., treat disease, confer improved qualities, expression of a protein of interest in a host cell, expression of a ribozyme, etc.), by one of ordinary skill in the art. Such nucleotide sequences include, but are not limited to, coding sequences of structural genes (e.g., reporter genes, selection marker genes, oncogenes, drug resistance genes, growth factors, etc.), and non-coding regulatory sequences which do not encode an mRNA or protein product (e.g., promoter sequence, polyadenylation sequence, termination sequence, enhancer sequence, etc.).

As used herein, the term “protein of interest” refers to a protein encoded by a nucleic acid of interest.

As used herein, the term “exogenous gene” refers to a gene that is not naturally present in a host organism or cell, or is artificially introduced into a host organism or cell.

The term “gene” refers to a nucleic acid (e.g., DNA or RNA) sequence that comprises coding sequences necessary for the production of a polypeptide or precursor (e.g., proinsulin). The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and includes sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ untranslated sequences. The sequences that are located 3′ or downstream of the coding region and which are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

As used herein, the terms “nucleic acid molecule encoding,” “DNA sequence encoding,” “DNA encoding,” “RNA sequence encoding,” and “RNA encoding” refer to the order or sequence of deoxyribonucleotides or ribonucleotides along a strand of deoxyribonucleic acid or ribonucleic acid. The order of these deoxyribonucleotides or ribonucleotides determines the order of amino acids along the polypeptide (protein) chain. The DNA or RNA sequence thus codes for the amino acid sequence.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The terms “homology” and “percent identity” when used in relation to nucleic acids refers to a degree of complementarity. There may be partial homology (i.e., partial identity) or complete homology (i.e., complete identity). A partially complementary sequence is one that at least partially inhibits a completely complementary sequence from hybridizing to a target nucleic acid sequence and is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe (i.e., an oligonucleotide which is capable of hybridizing to another oligonucleotide of interest) will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature” of a nucleic acid. The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation:

Tₘ=81.5+0.41 (% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42° C. in a solution consisting of 5×SSPE (43.8 μl NaCl, 6.9 μl NaH₂PO₄.H₂O and 1.85 μl EDTA, pH adjusted to 7.4 with NaOH), 0.5% SDS, 5×Denhardt's reagent and 100 μg/ml denatured salmon sperm DNA followed by washing in a solution comprising 0.1X SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 μl NaCl, 6.9 μl NaH₂PO₄.H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 μl NaCl, 6.9 g/l NaH₂PO₄.H₂O and 1.85 μl EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements are splicing signals, polyadenylation signals, termination signals, RNA export elements, internal ribosome entry sites, etc. (defined infra).

Transcriptional control signals in eukaryotes comprise “promoter” and “enhancer” elements. Promoters and enhancers consist of short arrays of DNA sequences that interact specifically with cellular proteins involved in transcription (Maniatis et al., Science 236:1237 [1987]). Promoter and enhancer elements have been isolated from a variety of eukaryotic sources including genes in yeast, insect and mammalian cells, and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types (for review see, Voss et al., Trends Biochem. Sci., 11:287 [1986]; and Maniatis et al., supra). For example, the SV40 early gene enhancer is very active in a wide variety of cell types from many mammalian species and has been widely used for the expression of proteins in mammalian cells (Dijkema et al., EMBO J. 4:761 [1985]). Two other examples of promoter/enhancer elements active in a broad range of mammalian cell types are those from the human elongation factor 1α gene (Uetsuki et al., J. Biol. Chem., 264:5791 [1989]; Kim et al., Gene 91:217 [1990]; and Mizushima and Nagata, Nuc. Acids. Res., 18:5322 [1990]) and the long terminal repeats of the Rous sarcoma virus (Gorman et al., Proc. Natl. Acad. Sci. USA 79:6777 [1982]) and the human cytomegalovirus (Boshart et al., Cell 41:521 [1985]).

As used herein, the term “promoter/enhancer” denotes a segment of DNA which contains sequences capable of providing both promoter and enhancer functions (i.e., the functions provided by a promoter element and an enhancer element, see above for a discussion of these functions). For example, the long terminal repeats of retroviruses contain both promoter and enhancer functions. The enhancer/promoter may be “endogenous” or “exogenous” or “heterologous.” An “endogenous” enhancer/promoter is one that is naturally linked with a given gene in the genome. An “exogenous” or “heterologous” enhancer/promoter is one that is placed in juxtaposition to a gene by means of genetic manipulation (i.e., molecular biological techniques such as cloning and recombination) such that transcription of that gene is directed by the linked enhancer/promoter.

The term “promoter,” “promoter element,” or “promoter sequence” as used herein, refers to a DNA sequence which when ligated to a nucleotide sequence of interest is capable of controlling the transcription of the nucleotide sequence of interest into mRNA. A promoter is typically, though not necessarily, located 5′ (i.e., upstream) of a nucleotide sequence of interest whose transcription into mRNA it controls, and provides a site for specific binding by RNA polymerase and other transcription factors for initiation of transcription.

Promoters may be constitutive or regulatable. The term “constitutive” when made in reference to a promoter means that the promoter is capable of directing transcription of an operably linked nucleic acid sequence in the absence of a stimulus (e.g., heat shock, chemicals, etc.). In contrast, a “regulatable” promoter is one that is capable of directing a level of transcription of an operably linked nucleic acid sequence in the presence of a stimulus (e.g., heat shock, chemicals, etc.), which is different from the level of transcription of the operably linked nucleic acid sequence in the absence of the stimulus.

As used herein the term, the term “in vitro” refers to an artificial environment and to processes or reactions that occur within an artificial environment. In vitro environments can consist of, but are not limited to, test tubes and cell cultures. The term “in vivo” refers to the natural environment (e.g., an animal or a cell) and to processes or reaction that occur within a natural environment.

### DETAILED DESCRIPTION OF THE INVENTION

The present invention is related to human stem cell lines comprising a targeted gene construct, and in particular to human embryonic stem cell lines comprising a reporter gene inserted into a desired locus via homologous recombination. In some embodiments, the human stem cell lines are human embryonic stem cell (hESC) lines. In some embodiments, the human stem cell lines remain pluripotent, maintain a normal karyotype, and allow for detection of gene expression of gene that is not expressed when the human stem cell line is in an undifferentiated state, but is expressed when differentiated into specific cell types. In some preferred embodiments, gene expression is detected by expression of inserted reporter gene. In some embodiments, the reporter gene encodes a fluorescent proteins and gene expression is detected by fluorescence microscopy or sorting by FACS.

In some preferred embodiments, the human stem cell lines comprise a reporter gene inserted into the Olig2 locus via homologous recombination. Olig2 is a transcription factor that plays a key role in motor neuron and oligodendrocyte differentiation in the central nervous system. Olig2⁺ cells in the pMN domain of the spinal cord generate motor neurons and oligodendrocytes, cell types that have the potential for treatment of spinal cord injury and other motor neuron disorders such as MS and ALS. Since Olig2 is important in the development of motor neurons and oligodendrocytes, the present invention provides a means to study differentiation of stem cells into motor neurons and oligodendrocytes, as well as the study of intrinsic and extrinsic factors that affect such differentiation. The human stem cell lines of the present invention also provide a means to study and determine optimal factors and conditions for cell differentiation.

A. Reporter Gene Constructs

In some embodiments, gene constructs comprising a nucleic acid encoding a reporter gene that are inserted or “knocked-in” a desired locus in a human stem cell. Methods for generating gene constructs for use in generating knock-in cell lines and the techniques for generating the cell lines are known to those of skill in the art, and may be found, for example, in Sambrook et al., 1989, Molecular Cloning, A Laboratory Manual, 3 ded., Cold Spring Harbor Laboratory; Yoo et al., 2003, Neuron, 37: 383; Watase et al., 2002, Neuron, 34:905; Lorenzetti et al., 2000, Human Molecular Genetics, 9:779; and Lin et al., 2001, Human Molecular Genetics, 10:137.

In some embodiments, the reporter gene constructs of the present invention comprise a nucleic acid sequence encoding a reporter gene flanked by a 5′ nucleic acid sequence that is homologous to a target gene of interest and a 3′ nucleic acid sequence that is homologous to the nucleic acid sequence of interest. In some embodiments, the reporter gene nucleic acid sequence is in frame with the 5′ and 3′ flanking sequence so that when the reporter gene construct is inserted into the target gene via homologous recombination, a fusion protein is expressed comprising a functional reporter gene product, such as green fluorescent protein.

The present invention is not limited to the use of any particular reporter gene nucleic acid sequence. Indeed, the use of a variety of reporter gene nucleic acid sequences is contemplated. In one embodiment, the present invention provides compositions and methods for transfecting stem cells with reporter genes encoding fluorescent proteins, wherein the expression of said proteins provide a viable means of following expression analysis in stem cells without having to sacrifice the cells. In some embodiments, the reporter genes encode fluorescent proteins, wherein the fluorescent proteins emit, for example, green, red, yellow, cyan (e.g., aqua or blue-green color), blue, yellow (GenBank Accession No. AY278271), orange (Genbank Accession No. AY278265), orange-red (GenBank Accession No. AY278270) or yellow-orange (GenBank Accession No. AY678267) fluorescence. Examples of fluorescent genes encoding for fluorescent proteins can be obtained commercially at, for example, Promega Corporation, Invitrogen, Clontech, Stratagene, BD Biosciences Pharmingen, Evrogen JSC. The fluorescent proteins originate from a variety of sources such as marine invertebrates, reef corals, and the like and include, but are not limited to, species from the genera Aqueorea, Montastrea (GenBank Accession No. AY218848), Zoanthus, Discosoma, and Heterectis. Additional examples of fluorescent proteins can be found at, for example, U.S. Pat. Nos. 6,884,620 and 6,645,761. The present invention is not limited by the fluorescent protein-encoding gene, and any fluorescent encoding gene from any origin is contemplated for use with the present invention. In some embodiments, the gene encoding the fluorescent protein has been optimized for mammalian expression by codon optimization for mammalian translational machinery.

In one embodiment, the present invention provides compositions and methods for transfecting stem cells with reporter genes encoding enzymatic proteins, wherein the expression of said proteins provide a viable means of following expression analysis in stem cells without having to sacrifice the cells. For example, the expression of reporter enzymatic proteins provide light output upon substrate utilization. Examples of such enzymatic proteins that produce light as a byproduct of substrate (e.g., luciferin, coelenterazine) utilization include, but are not limited to, luciferase, β-galactosidase and β-lactamase. Luciferase encoding reporter genes (e.g. Photinus sp. (GenBank Accession No. AY738222), Renilla sp. (GenBank Accession No. AF025844), Pyrophorus sp. (GenBank Accession No. AY258591 and AY258593), etc.) are commercially available from Promega Corporation, Stratagene and Clontech, for example. β-galactosidase encoding reporter genes are available from Promega Corporation and Clontech, and β-lactamase encoding reporter genes are available from Invitrogen, for example. In some embodiments, the luminescence producing reporter gene has been further optimized for mammalian expression by codon optimization for mammalian translational machinery.

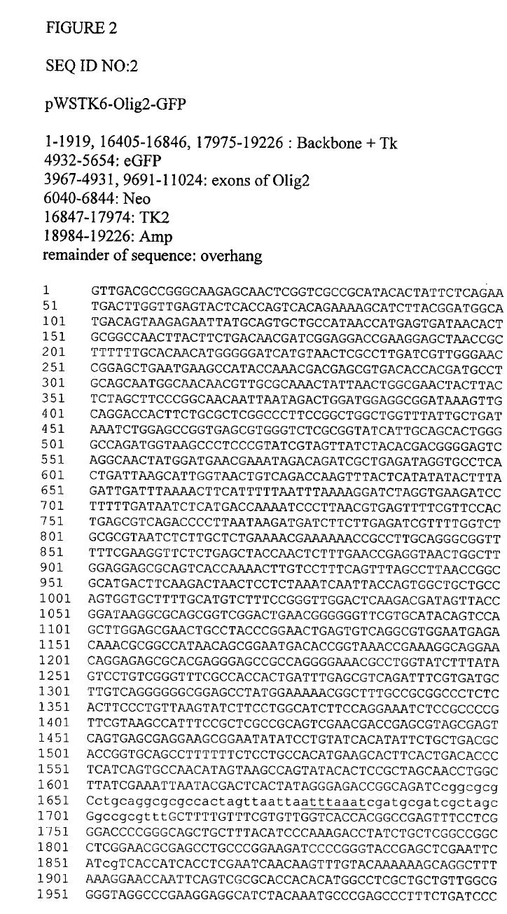

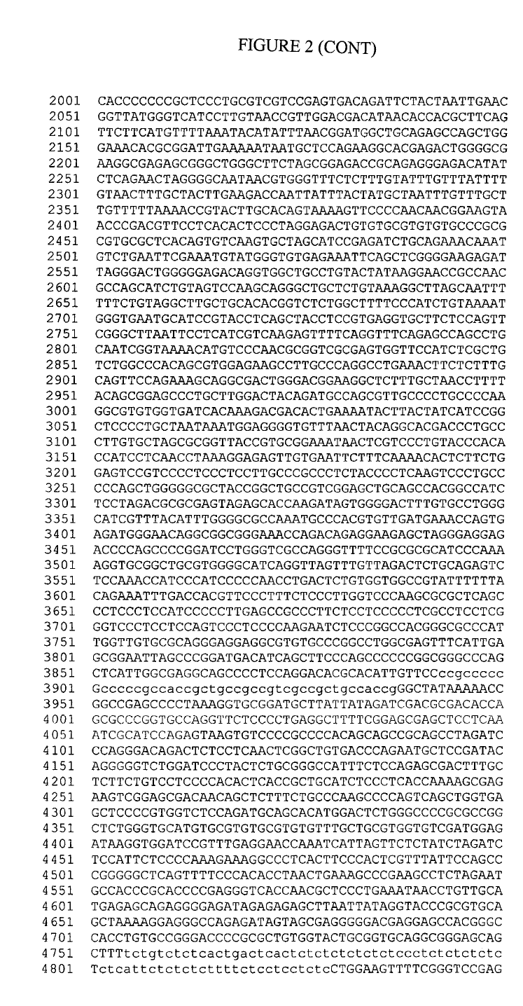

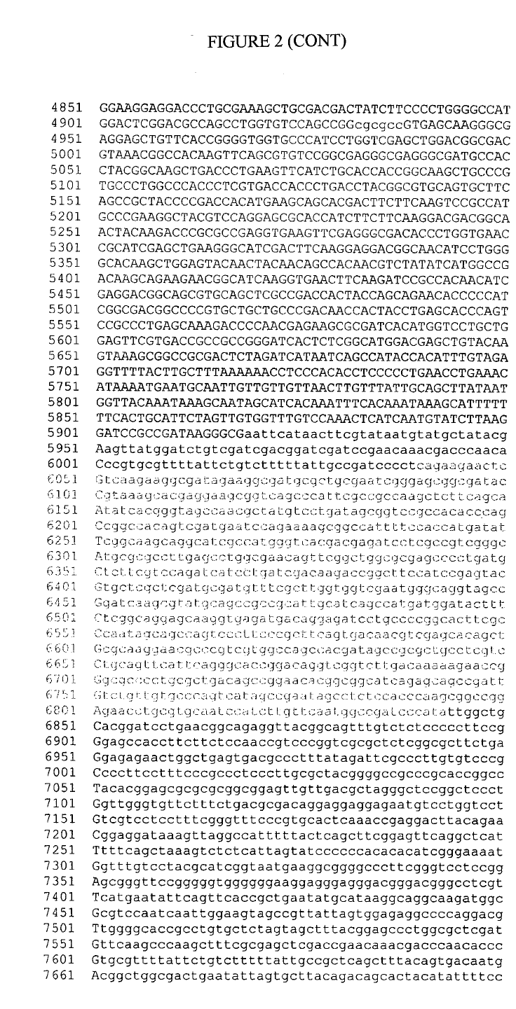

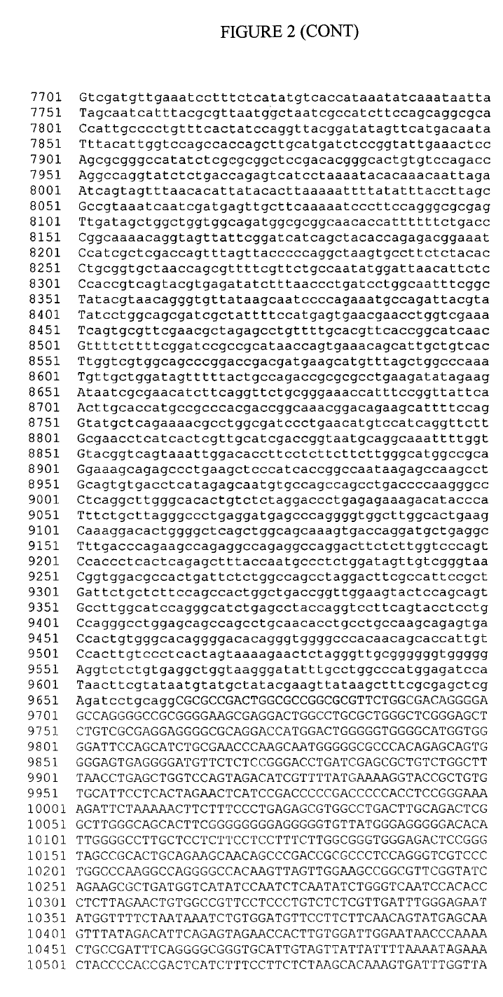

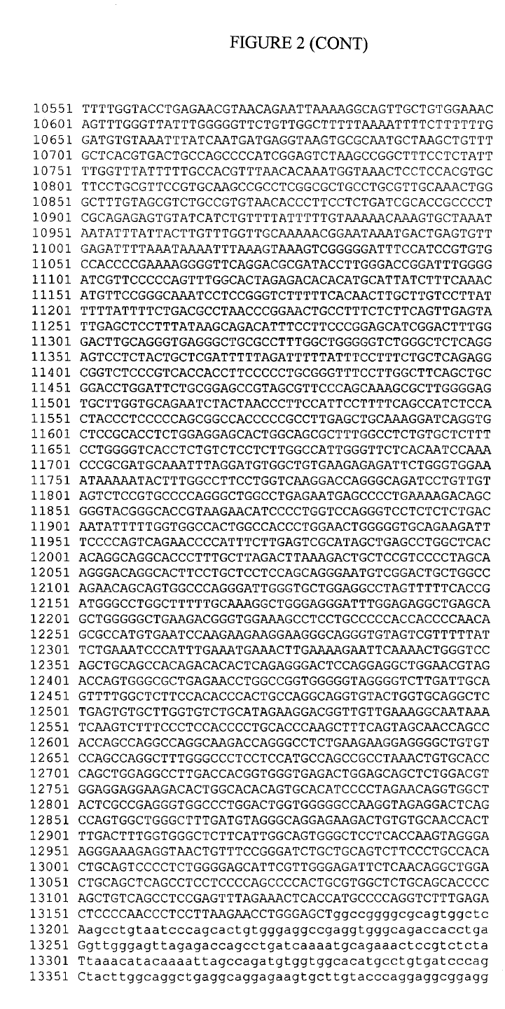

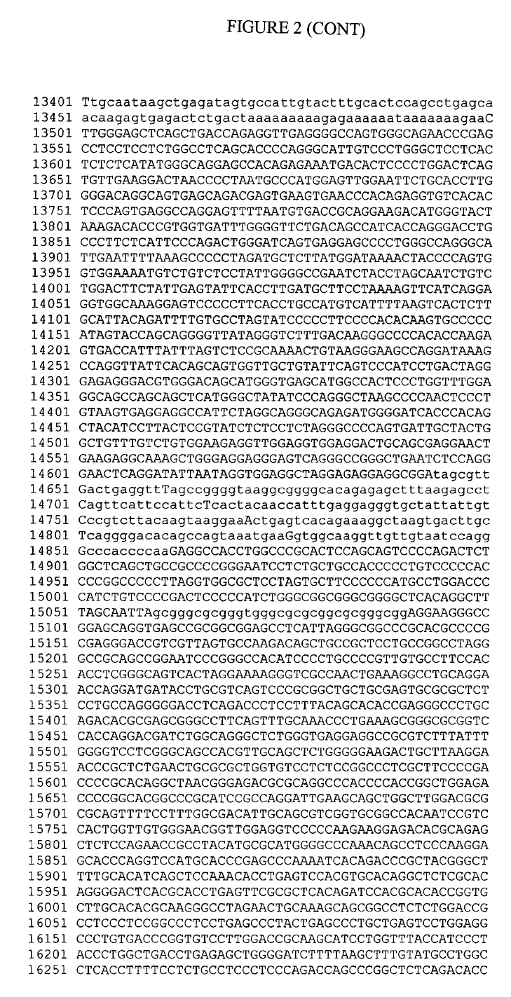

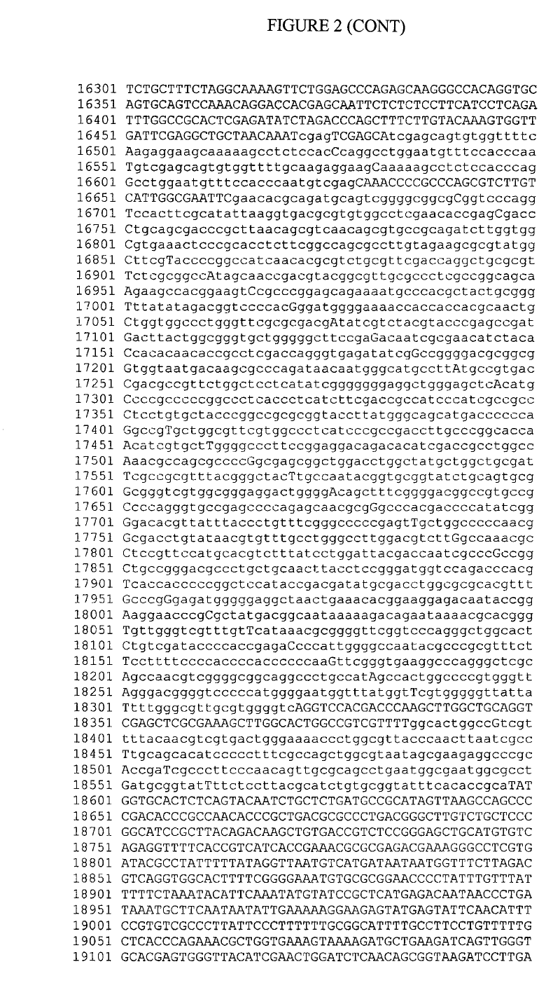

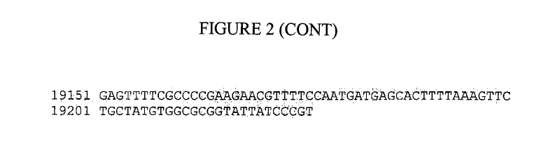

The present invention is not limited to the use of any particular 5′ and 3′ target gene flanking sequences. Indeed, the use of a variety of 5′ and 3′ target gene flanking sequences is contemplated. For example, a variety of different genes may be targeted and the gene may be targeted in different areas. In some preferred embodiments, the target gene is a gene that is not expressed or is substantially not expressed in undifferentiated stem cells. In some particular preferred embodiments, the target gene is human Olig2. Accordingly, in some preferred embodiments, the reporter gene construct comprises 5′ and 3′ flanking sequences that are homologous to human Olig2 [FIG. 2, SEQ ID NO: 1], wherein the flanking sequence flank a reporter gene nucleic acid sequence. In some preferred embodiments, the 5′ and 3′ flanking sequences are from about 5 to about 10 kilo bases in length and are from 95% to 100% homologous to the corresponding sequences in SEQ ID NO: 1. In some particularly preferred embodiments, the gene targeting constructs corresponds to SEQ ID NO:2.

In further preferred embodiments, the reporter gene constructs further comprise a selectable marker. The present invention is not limited to the use of any particular selectable marker. Indeed, the use of a variety of selectable markers is contemplated, including, but not limited to, dominant selectable markers such as the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. Other selectable markers are not dominant in that their use must be in conjunction with a cell line that lacks the relevant enzyme activity. Examples of non-dominant selectable markers include the thymidine kinase (tk) gene that is used in conjunction with tk⁻ cell lines, the CAD gene, which is used in conjunction with CAD-deficient cells, and the mammalian hypoxanthine-guanine phosphoribosyl transferase (hprt) gene, which is used in conjunction with hprt⁻ cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp. 16.9-16.15.

B. Stem Cell Lines

The present invention is not limited to the use of any particular type of human stem cells. Indeed, the use of a variety of types of human stem cells is contemplated. Methods for obtaining totipotent or pluripotent cells from humans, monkeys, mice, rats, pigs, cattle and sheep have been previously described. See, e.g., U.S. Pat. Nos. 5,453,357; 5,523,226; 5,589,376; 5,340,740; and 5,166,065 (all of which are specifically incorporated herein by reference); as well as, Evans, et al., Theriogenology 33(1): 125-128, 1990; Evans, et al., Theriogenology 33(1):125-128, 1990; Notarianni, et al., J. Reprod. Fertil. 41 (Suppl.):51-56, 1990; Giles, et al., Mol. Reprod. Dev. 36:130-138, 1993; Graves, et al., Mol. Reprod. Dev. 36:424-433, 1993; Sukoyan, et al., Mol. Reprod. Dev. 33:418-431, 1992; Sukoyan, et al., Mol. Reprod. Dev. 36:148-158, 1993; lannaccone, et al., Dev. Biol. 163:288-292, 1994; Evans & Kaufman, Nature 292:154-156, 1981; Martin, Proc Natl Acad Sci USA 78:7634-7638, 1981; Doetschmanet al. Dev Biol 127:224-227, 1988); Gileset al. Mol Reprod Dev 36:130-138, 1993; Graves & Moreadith, Mol Reprod Dev 36:424-433, 1993 and Bradley, et al., Nature 309:255-256, 1984.

Primate embryonic stem cells (ESCs) may be preferably obtained by the methods disclosed in U.S. Pat. Nos. 5,843,780 and 6,200,806, each of which is incorporated herein by reference. Primate (including human) stem cells may also be obtained from commercial sources such as WiCell, Madison, Wis. A preferable medium for isolation of ESCs is “ES medium.” ES medium consists of 80% Dulbecco's modified Eagle's medium (DMEM; no pyruvate, high glucose formulation, Gibco BRL), with 20% fetal bovine serum (FBS; Hyclone), 0.1 mM β-mercaptoethanol (Sigma), 1% non-essential amino acid stock (Gibco BRL). Preferably, fetal bovine serum batches are compared by testing clonal plating efficiency of a low passage mESC line (ESⱼₜ₃), a cell line developed just for the purpose of this test. FBS batches must be compared because it has been found that batches vary dramatically in their ability to support embryonic cell growth, but any other method of assaying the competence of FBS batches for support of embryonic cells will work as an alternative.

Primate ESCs are isolated on a confluent layer of murine embryonic fibroblast in the presence of ES medium. Embryonic fibroblasts are preferably obtained from 12 day old fetuses from outbred CF₁ mice (SASCO), but other strains may be used as an alternative. Tissue culture dishes are preferably treated with 0.1% gelatin (type I; Sigma). Recovery of rhesus monkey embryos has been demonstrated, with recovery of an average 0.4 to 0.6 viable embryos per rhesus monkey per month, Seshagiri et al. Am J Primatol 29:81-91, 1993. Embryo collection from marmoset monkey is also well documented (Thomson et al. “Non-surgical uterine stage preimplantation embryo collection from the common marmoset,” J Med Primatol, 23:333-336 (1994)). Here, the zona pellucida is removed from blastocysts by brief exposure to pronase (Sigma). For immunosurgery, blastocysts are exposed to a 1:50 dilution of rabbit anti-marmoset spleen cell antiserum (for marmoset blastocysts) or a 1:50 dilution of rabbit anti-rhesus monkey (for rhesus monkey blastocysts) in DMEM for 30 minutes, then washed for 5 minutes three times in DMEM, then exposed to a 1:5 dilution of Guinea pig complement (Gibco) for 3 minutes.

After two further washes in DMEM, lysed trophectoderm cells are removed from the intact inner cell mass (ICM) by gentle pipetting, and the ICM plated on mouse inactivated (3000 rads gamma irradiation) embryonic fibroblasts. After 7-21 days, ICM-derived masses are removed from endoderm outgrowths with a micropipette with direct observation under a stereo microscope, exposed to 0.05% Trypsin-EDTA (Gibco) supplemented with 1% chicken serum for 3-5 minutes and gently dissociated by gentle pipetting through a flame polished micropipette.

Dissociated cells are replated on embryonic feeder layers in fresh ES medium, and observed for colony formation. Colonies demonstrating ESC-like morphology are individually selected, and split again as described above. The ESC-like morphology is defined as compact colonies having a high nucleus to cytoplasm ratio and prominent nucleoli. Resulting ESCs are then routinely split by brief trypsinization or exposure to Dulbecco's Phosphate Buffered Saline (without calcium or magnesium and with 2 mM EDTA) every 1-2 weeks as the cultures become dense. Early passage cells are also frozen and stored in liquid nitrogen.

In some embodiments, cord blood stem cells or umbilical cord matrix stem cells are utilized. Sources of such stem cells are known in the art. Brunstein et al., Br. J. Haematol. 137(1):20-35 (2007); Sun et al., Biochem. Biophys. Res. Commun. 354(4):919-23 (2007); McGuckin et al., Acta Neurobiol. Exp. 66(4):321-9 (2006); Riodan et al., Transl. Med. 30:5-8 (2007); Weiss et al., Stem Cell Rev. 2(2):155-62 (2006).

In some embodiments, the reporter gene constructs described above are introduced into a desired cell line. The present invention is not limited by the method of introduction. In some preferred embodiments, the reporter gene constructs are introduced into a desired stem cell line by electroporation. In some embodiments, the conditions utilized are described in Example 2 below. In some preferred embodiments, the electroporation is conducted under conditions of low voltage and low capacitance. In some preferred embodiments, the voltage ranges from about 150V to about 350 V, and most preferably is about 250 V. In some preferred embodiments, the capacitance ranges from about 150 to 350 μF, most preferably about 250 μF. In some embodiments, the stem cell line is cultured without feeder cells prior to electroporation. In some embodiments, the stem cell line is plated onto feeder cell line, preferably mouse embryonic fibroblasts, following electroporation. In some embodiments, the stem cell line is harvested with an enzyme after electroporation, preferably Accutase™. Following electroporation, in preferred embodiments, clones containing the reporter gene construct are selected using an antibiotic, preferably G418. In preferred embodiments, the G418 is at a dosage ranging from about 50 to 200 μg/ml.

In preferred embodiments, the reporter gene construct is introduced into a gene that is not expressed in undifferentiated stem cells. As described in the examples below, the fact that the reporter gene is not expressed in undifferentiated stem cells complicates selection of clones and development of cell lines that correctly express the reporter gene because the cells must be induced to differentiation prior to identification of expression of the reporter gene. It is believed that the exemplified human stem cell lines are the first human stem cell lines to contain a targeted insertion in a gene that is not expressed in undifferentiated stem cells. The methods described herein may be used to target other desired genes that are not expressed in undifferentiated stem cells. In preferred embodiments, the cells lines of the present invention remain pluripotent or totipotent, have a normal karyotype, and allow visualization of the reporter gene by a method such as fluorescence microscopy or fluorescence activated cell sorting. In preferred embodiments, expression of the reporter gene serves as a marker for differentiation of the cell into a particular cell type. In preferred embodiments, where Olig2 is targeted, expression of the marker gene occurs during differentiation into motor neurons and oligodendrocytes.

C. Use of Cell Lines

The cell lines of the present invention have a variety of uses. In particular, the cell lines of the present invention are useful for screening for compounds that are useful for modulating the differentiation of human stem cells into a desired cell type, such as oligodendrocytes and motor neurons, for identifying compounds that may be useful in the regeneration or growth of cell types such as motor neurons and oligodendrocytes so that spinal cord injuries and diseases or disorders of the central nervous system may be treated, and for screening for compounds that are neurotoxic.

Accordingly, in some embodiments, the present invention provides screening assays. The screening methods of the present invention utilize stem cell lines containing a reporter gene inserted into a locus of interest, such as the olig2 locus. For example, in some embodiments, the present invention provides methods of screening for compounds that alter (e.g., either block or trigger) the expression of a reporter gene inserted into a locus of interest. The compounds or agents may interfere with transcription, by interacting, for example, with the promoter region of the gene contained within the locus of interest. Alternatively, the compounds or agents may interfere with pathways that are upstream or downstream of the biological activity of the locus of interest.

In one screening method, candidate compounds are evaluated for their ability to affect expression of the reporter gene inserted into the locus of interest. In preferred embodiments, the gene in the locus of interest is a gene that is not expressed in undifferentiated stem cells. In some embodiments, the effect of candidate compounds on expression of the gene in the locus of interest is assayed for by detecting the level of fluoresence of a cells containing the reporter gene construct. In other embodiments, mRNA expression can be detected by any suitable method.

Specifically, the present invention provides screening methods for identifying modulators, i.e., candidate or test compounds or agents (e.g., proteins, peptides, peptidomimetics, peptoids, small molecules or other drugs) which bind to promoters upstream of the inserted reporter construct or that interact with proteins or the expression of proteins that bind to promoters upstream of the inserted reporter construct, that have an inhibitory (or stimulatory) effect on, for example, expression of the inserted reporter construct. Compounds thus identified can be used to modulate the activity of target gene products (e.g., Olig2) either directly or indirectly in a therapeutic protocol, to elaborate the biological function of the target gene product, or to identify compounds that disrupt normal target gene interactions. Compounds that modulate the activity or expression of Olig2, for example, are useful in the treatment of diseases associated with functioning of the central nervous system and for use in treating damage to motor neurons and oligodendrocytes.

In one embodiment, the invention provides assays for screening candidate or test compounds that alter the expression of a gene of interest, such as Olig2, that expressed during differentiation of a particular cell type, for example motor neurons or oligodendrocytes. In another embodiment, the invention provides assays for screening candidate or test compounds that bind to or modulate the activity of proteins that alter the expression of a gene of interest such as Olig2.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]).

In one embodiment, an assay is a cell-based assay in which a cell that expresses a reporter gene inserted into a locus of interest is contacted with a test compound, and the ability of the test compound to the modulate expression of the reporter gene is determined. Determining the ability of the test compound to modulate reporter gene expression can be accomplished by monitoring, for example, changes in fluorescence, luminescence, color, or the like. Suitable monitoring methods, include, but are not limited to, fluorescence activated cell sorting (FACS) and fluorescence microscopy.

In some other embodiments, the cell of the present invention are used for toxological testing. In some embodiments, the cells of the present invention are contacted with a compound suspected of being a toxin and expression of the reporter gene is modified. In some preferred embodiments, the compounds are suspected neurotoxins.

In some embodiments, the cells of the present invention are used in high throughput screening assays. In some embodiments, the high throughput screening assays are used for identification of potential drugs. In some preferred embodiments, the cells of the present invention are used to screen for drugs that are useful for treating neurological conditions and for treating damage to components of the central nervous system such as motor neurons and oligidendrocytes.

In further embodiments, the cells of the present invention are used to monitor responses to intrinsic or extrinsic factors during differentiation of a target cell type. In some preferred embodiments, the target cell type is a motor neuron or oligodendrocyte. In some embodiments, the cell of the present invention are utilized to identify optimal conditions for developing cells of a desired cell type, such as motor neurons or oligodendrocytes.

### EXAMPLES

### Example 1

### Generation of hOlig2-EGFP Knock-in Vector

A human BAC clone containing the Olig2 gene was purchased from Invitrogen (Cat# RP11-585D4). The targeting vector was constructed in DH50α using homologous recombination. The translational start codon of hOlig2 was designated as the +1 position is used throughout to describe the Olig2 gene. To generate the targeting construct, pENTR1a (Invitrogen) was modified to grow in DH5α as a low-copy plasmid, renamed to pStartK and was used as the template to amplify the fragment outside attL1 and attL2. The primers contained two overhangs that were homologous to the flanking sequence of hOlig2, so that when this PCR product was transformed into the ET competent hOlig2 BAC, full-length hOlig2 gene and ˜2 kb of its upstream and ˜5.3 kb of its downstream sequences were pulled out into pStartK as a result of homologous recombination as selected by ampcillin. The resultant plasmid was named pStartK-hOlig2 and its sequence was verified by limited sequencing. Then, a fragment containing a total of 100 bp homology arm of hOlig2 exon 2 (−19 to +30 and +976 to +1025, respectively), two AscI sites and cat, the gene encoding chloramphenicol was amplified and co-transfected with pStartK-hOlig2 into ET competent DH5α, seeking to replace the hOlig2 exon 2 by cat, and the resultant plasmid was selected by chloramphenicol and ampcillin. The positive plasmid was verified by partial sequencing and named pStartK-hOlig2cam. pStartK-hOlig2cam was then digested by AscI and an EGFP-neomycin fragment was purified and ligated into pStartK-hOlig2cam, resulting pStartK-hOlig2eGFP, in which cat was swapped by sequence encoding EGFP and neomycin (expression of neomycin was driven by RNA Pol II promoter). In order to add a negative selection site to this vector, pStartK-hOlig2eGFP was incubated with a Gene gateway (Invitrogen) plasmid which contained attR1 and attR2 sites and Tk2, a thymidine kinase gene. After incubation with clonase (Invitrogen), the hOlig2eGFP fragment was exchanged via LR recombination and was ligated with Tk2 gene. The final construct was selected by ampicillin and named pWSTK3 hOlig2eGFP. When delivered into hESCs, only homologous recombinants would have Tk2 gene excised and survive under negative selection drug 6-TG or FIAU. The sequence of the targeting vectors is provided in FIG. 2 (SEQ ID NO:2).

### Example 2

### Generation of Olig2-GFP Knock-in Clones in Human Embryonic Stem Cell Line BG01

BG01 hESC line was maintained on a layer of mitomycin C (Sigma) inactivated mouse embryonic fibroblast cells (MEF) in hESC medium containing DMEM-F12, 20% Knockout Serum, 1% Non-essential amino acid, 55 mM 2-Mercaptoethanol, 2 mM L-Glutamine (all above from Invitrogen), supplemented with 4 ng/ml basic FGF (Peprotech), or on Matrigel (BD Biosciences) coated dishes in medium conditioned with MEF for 24 hours.

To generate Olig2-GFP reporter KI lines, a total of 5×10⁶ to 1×10⁷ BG01 cells were dissociated using accutase (Sigma) and incubated with 30 μg of linearized pWSTK3 hOlig2eGFP (targeting vector). Mixture of DNA and cells was then transferred to a 4 mm cuvette and electroporated using a Bio-Rad Xcell Total system for a single pulse of 250V, 250 μF. Electroporated cells were plated onto MEF layers for recovery. Seventy two hours post-transfection, G418 (50 μg/ml for the first 3-4 days and increased to 200 μg/ml after one week) (Invitrogen) and FIAU (125 nM, Maravek Biochmicals) were added to medium everyday. Resistant clones were manually picked after 21 days of double selection and plated on MEF feeder layers or Matrigel coated dishes for further expansion.

Southern Blot analysis was used to identify the targeted clones. A total of 106 clones were obtained as described above from which genomic DNA was extracted. HindIII-digested genomic DNA from each clone was hybridized to a probe derived from the 5′ of the homology arm by Southern blot analysis. Six clones were confirmed to be correctly targeted to the Olig2 site. Positive clones of Olig2-GFP knock-in hESCs were maintained in hESC medium containing 100-200 mg/ml G418. Cells were karyotyped routinely (Cell Line Genetics, Madison, Wis.). GFP-positive cells were visible under fluorescence microscopy after 17 days of differentiation under conditions favored for motor neuron and oligodendrocyte formation.

For differentiation studies, Olig2-GFP knock-in clones were maintained in hESC medium and detached by using collagenase IV (1 mg/ml, Invitrogen). Cells were triturated to make small clumps, which were then transferred to Ultralow attachment dishes (Corning) to form EBs. EBs were cultured in suspension for 5 days in MDFK medium (DMEM-F12, 5% Knockout Serum, 1×ITS-X,1% Non-essential amino acid, 55 mM 2-Mercaptoethanol, 2 mM L-Glutamine, all from Invitrogen). EBs were further induced to express Olig2-GFP by adding 2 μM all-trans-retinoic acid (RA, Sigma) and 30 nM recombinant sonic hedgehog (Shh, R&D systems) every other day for an additional 10-20 days. EBs generated from these Olig2-GFPKI clones were then examined under fluorescence microscope for the expression of GFP and co-labeling of Olig2 and GFP by performing immunocytochemistry of Olig2 antibody staining.

### Example 3

### Transfection of Human Embryonic Stem Cells

Human embryonic stem cells (hESCs) are difficult to transfect due to the growth habits of the cells. hESCs tend to grow in clumps and in general difficult to form from singles cells as opposed to mouse stem cells which can grow from single cells. Homologous recombination in hESCs seems to be extremely difficult to achieve as to date, only two genes, Oct4 and HPRT, have been targeted in hESCs. Both of these genes are expressed in hESCs, which aids in selection of transformants even when transformation efficiency is low. Even with this advantage, results with hESCs stand in sharp contrast to mouse ESCs wherein hundreds of genes have been successfully targeted by homologous recombination. Yates and Daley, Gene Ther. 13(20):1431-9 (2006), report that HR (homologous recombination) has not been as extensively employed in hESCs (compared to mESCs), and to date only three HR-targeted hESC lines (2 HPRT lines and one Oct4 line) exist. The poor results are partly due to the major difficulties encountered in transfecting DNA into hESCs. Zwaka and Thomson, Nat. Biotechnol. 21(3):319-21 (2003) report that although the targeting frequency for Oct4 and HPRT1 genes is similar to the results obtained in mESCs, it will be important to determine whether this similarity of rates between human and mESCs holds true for genes not expressed in ESCs.

To date, no genes that are not expressed in hESCs have been targeted except for the work described herein. To overcome the difficulties inherent in targeting genes that are not expressed in hESCs, several approaches were utilized to optimize transfection efficiency and targeting efficiency. These approaches included chemical transfection including lipid mediated transfection reagents such as Lipofectamine 2000 and FuGene, Amaxa nucleofactor (nucleoporation), and electroporation (Bio-Rad Xcell total system). Neither chemical transfection nor nucleoporation generated any targeted clones. Optimized electroporation conditions were then utilized and resulted in 6 targeted clones with a transfection efficiency of approximately 10⁻⁸-10⁻⁷ after eight rounds of electroporation.

The following Parameters were Tested:

Culture methods: It was necessary to determine whether the hESCs should be grown on mouse embryonic fibroblast (MEF) feeders or on matrix without feeder cells. It was found that hESCs cultured without feeders prior to electroporation and plated on a layer of MEFs after electroporation gave the highest transfection efficiency as well as targeting efficiency. This result has not been reported previously.

Cell harvest: It was also necessary to determine what method should be used to harvest cells prior to electroporation. A number of different enzymes were tested, including trypsin (the most commonly used enzyme to detach cells—this is also the enzyme used to passage mouse ESCs), collagenase IV (often used for hESC passaging), and Accutase™. The only enzyme used which resulted in targeted clones was accutase.

Electroporation conditions: It was also necessary to test different electroporation conditions. Five conditions were tested: 800V, 10 mF; 250V, 250 mF; 500V, 500 mF; 250V, 250 mF; 320V, 200 mF. Among these, high voltage such as 800V, or high voltage high capacitance such as 500V, 500 mF, which usually work for mouse ESCs, did not work in hESCs. However, low voltage and low capacitance such as 250V, 250 mF gave the highest transfection efficiency and targeting efficiency. Indeed, all the targeted clones were obtained from electroporation using lower voltage and low capacitance.

Dosage for drug selections: It was also necessary to determine proper dosages for selection of target clones. Here again, the dosages work for mouse ESCs (e.g. 500-1000 μg/ml G418) did not work for hESCs (For G418 selection, we gradually increased the drug dosage from 50 to 200 μg/ml). Higher concentrations of drug killed all hESCs.

Expanding and maintaining transfected clones: It was also necessary to develop methods for expanding and maintaining transfected clones. It was found that a combination of enzymatic and manual dissection was best to obtain and maintain transfected clones.

1. A human stem cell comprising a reporter gene targeted into a gene that is not expressed in said human stem cell prior to differentiation.

2. The human stem cell of claim 1, wherein said human stem cell is an embryonic stem cell.

3. The human stem cell of claim 1, wherein said reporter gene is targeted into olig2 locus.

4. The human stem cell of claim 1, wherein said reporter gene encodes a fluorescent protein.

5. The human stem cell of claim 1, wherein said fluorescent protein is selected from the group consisting of green fluorescent protein, red fluorescent protein, yellow fluorescent protein.

6. The human stem cell of claim 1, wherein said reporter gene is targeted by homologous recombination.

7. An isolated stem cell line comprising the human stem cells of claim 1.

8. An in vitro cell culture comprising the human stem cells of claim 1.

9. An organism comprising the human stem cells of claim 1.

10. A tissue comprising the human stem cells of claim 1.

11. A method of screening compounds comprising:
a) providing human stem cells comprising a reporter gene targeted into a gene that is not expressed in said human stem cells prior to differentiation;
b) contacting said human stem cells with said at least one compound;
c) culturing said human stem cells; and
d) assaying said human stem cells for expression of said reporter gene.

12. The method of claim 11, wherein said assaying is performed by a method selected from the group consisting of fluorescence microscopy and fluorescence activated cell sorting.

13. The method of claim 11, wherein said culturing comprises culturing said human stem cells under conditions such that said human stem cell is allowed to differentiate.

14. The method of claim 11, wherein a plurality of cells are provided and said contacting step comprises contacting said plurality of cells with a library of compounds in a high throughput assay.

15. The method of claim 11, wherein said compound is selected from the group consisting of proteins, peptides, polypeptides and small organic compounds.

16. The method of claim 11, wherein said human stem cells are embryonic stem cells.

17. The method of claim 11, further comprising the step of selecting a compound.

18. The method of claim 17, further comprising the step of testing said compound in a human animal.

19. The method of claim 18, further comprising the step of packaging said compound for use in animals.

20. A method of screening for neurotoxins comprising providing:
a) providing human stem cells comprising a reporter gene inserted into the Olig2 locus;
b) contacting said human stem cells with said at least one compound suspected of being a neurotoxin; and
c) assaying said human stem cells for expression of said reporter gene.

21. The method of claim 20, wherein said assaying is performed by a method selected from the group consisting of fluorescence microscopy and fluorescence activated cell sorting.

22. The method of claim 20, wherein said human stem cells are embryonic stem cells.


SEQUENCE LISTING (2 sequences)

SEQ ID NO: 1 - DNA, 3362 bp, Homo sapiens
gtgcggatgcttattatagatcgacgcgacaccagcgcccggtgccaggttctcccctgaggcttttcggagcgagctcctcaaatcgcatccagagtaagtgtccccgccccacagcagccgcagcctagatcccagggacagactctcctcaactcggctgtgacccagaatgctccgatacagggggtctggatccctactctgcgggccatttctccagagcgactttgctcttctgtcctccccacactcaccgctgcatctccctcaccaaaagcgagaagtcggagcgacaacagctctttctgcccaagccccagtcagctggtgagctccccgtggtctccagatgcagcacatggactctgggccccgcgccggctctgggtgcatgtgcgtgtgcgtgtgtttgctgcgtggtgtcgatggagataaggtggatccgtttgaggaaccaaatcattagttctctatctagatctccattctccccaaagaaaggccctcacttcccactcgtttattccagcccgggggctcagttttcccacacctaactgaaagcccgaagcctctagaatgccacccgcaccccgagggtcaccaacgctccctgaaataacctgttgcatgagagcagaggggagatagagagagcttaattataggtacccgcgtgcagctaaaaggagggccagagatagtagcgagggggacgaggagccacgggccacctgtgccgggaccccgcgctgtggtactgcggtgcaggcgggagcagcttttctgtctctcactgactcactctctctctctctccctctctctctctctcattctctctcttttctcctcctctcctggaagttttcgggtccgagggaaggaggaccctgcgaaagctgcgacgactatcttcccctggggccatggactcggacgccagcctggtgtccagccgcccgtcgtcgccagagcccgatgacctttttctgccggcccggagtaagggcagcagcggcagcgccttcactgggggcaccgtgtcctcgtccaccccgagtgactgcccgccggagctgagcgccgagctgcgcggcgctatgggctctgcgggcgcgcatcctggggacaagctaggaggcagtggcttcaagtcatcctcgtccagcacctcgtcgtctacgtcgtcggcggctgcgtcgtccaccaagaaggacaagaagcaaatgacagagccggagctgcagcagctgcgtctcaagatcaacagccgcgagcgcaagcgcatgcacgacctcaacatcgccatggatggcctccgcgaggtcatgccgtacgcacacggcccttcggtgcgcaagctttccaagatcgccacgctgctgctggcgcgcaactacatcctcatgctcaccaactcgctggaggagatgaagcgactggtgagcgagatctacgggggccaccacgctggcttccacccgtcggcctgcggcggcctggcgcactccgcgcccctgcccgccgccaccgcgcacccggcagcagcagcgcacgccgcacatcaccccgcggtgcaccaccccatcctgccgcccgccgccgcagcggctgctgccgccgctgcagccgcggctgtgtccagcgcctctctgcccggatccgggctgccgtcggtcggctccatccgtccaccgcacggcctactcaagtctccgtctgctgccgcggccgccccgctggggggcgggggcggcggcagtggggcgagcgggggcttccagcactggggcggcatgccctgcccctgcagcatgtgccaggtgccgccgccgcaccaccacgtgtcggctatgggcgccggcagcctgccgcgcctcacctccgacgccaagtgagccgactggcgccggcgcgttctggcgacaggggagccaggggccgcggggaagcgaggactggcctgcgctgggctcgggagctctgtcgcgaggaggggcgcaggaccatggactgggggtggggcatggtggggattccagcatctgcgaacccaagcaatgggggcgcccacagagcagtggggagtgaggggatgttctctccgggacctgatcgagcgctgtctggctttaacctgagctggtccagtagacatcgttttatgaaaaggtaccgctgtgtgcattcctcactagaactcatccgacccccgacccccacctccgggaaaagattctaaaaacttctttccctgagagcgtggcctgacttgcagactcggcttgggcagcacttcggggggggagggggtgttatgggagggggacacattggggccttgctcctcttcctcctttcttggcgggtgggagactccgggtagccgcactgcagaagcaacagcccgaccgcgccctccagggtcgtccctggcccaaggccaggggccacaagttagttggaagccggcgttcggtatcagaagcgctgatggtcatatccaatctcaatatctgggtcaatccacaccctcttagaactgtggccgttcctccctgtctctcgttgatttgggagaatatggttttctaataaatctgtggatgttccttcttcaacagtatgagcaagtttatagacattcagagtagaaccacttgtggattggaataacccaaaactgccgatttcaggggcgggtgcattgtagttattattttaaaatagaaactaccccaccgactcatctttccttctctaagcacaaagtgatttggttattttggtacctgagaacgtaacagaattaaaaggcagttgctgtggaaacagtttgggttatttgggggttctgttggctttttaaaattttcttttttggatgtgtaaatttatcaatgatgaggtaagtgcgcaatgctaagctgtttgctcacgtgactgccagccccatcggagtctaagccggctttcctctattttggtttatttttgccacgtttaacacaaatggtaaactcctccacgtgcttcctgcgttccgtgcaagccgcctcggcgctgcctgcgttgcaaactgggctttgtagcgtctgccgtgtaacacccttcctctgatcgcaccgcccctcgcagagagtgtatcatctgttttatttttgtaaaaacaaagtgctaaataatatttattacttgtttggttgcaaaaacggaataaatgactgagtgttgagattttaaataaaatttaaagtaaagtcgggggatttccatccgtgtgccaccccgaaaaggggttcaggacgcgataccttgggaccggatttggggatcgttcccccagtttggcacta

SEQ ID NO: 2 - DNA, 19176 bp, Homo sapiens
gttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcaacctggcttatcgaaattaatacgactcactatagggagaccggcagatccggcgcgcctgcaggcgcgccactagttaattaatttaaatcgatgcgatcgctagcggccgcgtttgcttttgtttcgtgttggtcaccacggccgagtttcctcgggaccccgggcagctgctttacatcccaaagacctatctgctcggccggcctcggaacgcgagcctgcccggaagatccccgggtaccgagctcgaattcatcgtcaccatcacctcgaatcaacaagtttgtacaaaaaagcaggctttaaaggaaccaattcagtcgcgcaccacacatggcctcgctgctgttggcggggtaggcccgaaggaggcatctacaaatgcccgagccctttctgatccccacccccccgctccctgcgtcgtccgagtgacagattctactaattgaacggttatgggtcatccttgtaaccgttggacgacataacaccacgcttcagttcttcatgttttaaatacatatttaacggatggctgcagagccagctgggaaacacgcggattgaaaaataatgctccagaaggcacgagactggggcgaaggcgagagcgggctgggcttctagcggagaccgcagagggagacatatctcagaactaggggcaataacgtgggtttctctttgtatttgtttattttgtaactttgctacttgaagaccaattatttactatgctaatttgtttgcttgtttttaaaaccgtacttgcacagtaaaagttccccaacaacggaagtaacccgacgttcctcacactccctaggagactgtgtgcgtgtgtgcccgcgcgtgcgctcacagtgtcaagtgctagcatccgagatctgcagaaacaaatgtctgaattcgaaatgtatgggtgtgagaaattcagctcggggaagagattagggactgggggagacaggtggctgcctgtactataaggaaccgccaacgccagcatctgtagtccaagcagggctgctctgtaaaggcttagcaattttttctgtaggcttgctgcacacggtctctggcttttcccatctgtaaaatgggtgaatgcatccgtacctcagctacctccgtgaggtgcttctccagttcgggcttaattcctcatcgtcaagagttttcaggtttcagagccagcctgcaatcggtaaaacatgtcccaacgcggtcgcgagtggttccatctcgctgtctggcccacagcgtggagaagccttgcccaggcctgaaacttctctttgcagttccagaaagcaggcgactgggacggaaggctctttgctaaccttttacagcggagccctgcttggactacagatgccagcgttgcccctgccccaaggcgtgtggtgatcacaaagacgacactgaaaatacttactatcatccggctcccctgctaataaatggaggggtgtttaactacaggcacgaccctgcccttgtgctagcgcggttaccgtgcggaaataactcgtccctgtacccacaccatcctcaacctaaaggagagttgtgaattctttcaaaacactcttctggagtccgtcccctccctccttgcccgccctctacccctcaagtccctgcccccagctgggggcgctaccggctgccgtcggagctgcagccacggccatctcctagacgcgcgagtagagcaccaagatagtggggactttgtgcctgggcatcgtttacatttggggcgccaaatgcccacgtgttgatgaaaccagtgagatgggaacaggcggcgggaaaccagacagaggaagagctagggaggagaccccagccccggatcctgggtcgccagggttttccgcgcgcatcccaaaaggtgcggctgcgtggggcatcaggttagtttgttagactctgcagagtctccaaaccatcccatcccccaacctgactctgtggtggccgtattttttacagaaatttgaccacgttccctttctcccttggtcccaagcgcgctcagccctccctccatcccccttgagccgcccttctcctccccctcgcctcctcgggtccctcctccagtccctccccaagaatctcccggccacgggcgcccattggttgtgcgcagggaggaggcgtgtgcccggcctggcgagtttcattgagcggaattagcccggatgacatcagcttcccagccccccggcgggcccagctcattggcgaggcagcccctccaggacacgcacattgttccccgcccccgcccccgccaccgctgccgccgtcgccgctgccaccgggctataaaaaccggccgagcccctaaaggtgcggatgcttattatagatcgacgcgacaccagcgcccggtgccaggttctcccctgaggcttttcggagcgagctcctcaaatcgcatccagagtaagtgtccccgccccacagcagccgcagcctagatcccagggacagactctcctcaactcggctgtgacccagaatgctccgatacagggggtctggatccctactctgcgggccatttctccagagcgactttgctcttctgtcctccccacactcaccgctgcatctccctcaccaaaagcgagaagtcggagcgacaacagctctttctgcccaagccccagtcagctggtgagctccccgtggtctccagatgcagcacatggactctgggccccgcgccggctctgggtgcatgtgcgtgtgcgtgtgtttgctgcgtggtgtcgatggagataaggtggatccgtttgaggaaccaaatcattagttctctatctagatctccattctccccaaagaaaggccctcacttcccactcgtttattccagcccgggggctcagttttcccacacctaactgaaagcccgaagcctctagaatgccacccgcaccccgagggtcaccaacgctccctgaaataacctgttgcatgagagcagaggggagatagagagagcttaattataggtacccgcgtgcagctaaaaggagggccagagatagtagcgagggggacgaggagccacgggccacctgtgccgggaccccgcgctgtggtactgcggtgcaggcgggagcagcttttctgtctctcactgactcactctctctctctctccctctctctctctctcattctctctcttttctcctcctctcctggaagttttcgggtccgagggaaggaggaccctgcgaaagctgcgacgactatcttcccctggggccatggactcggacgccagcctggtgtccagccggcgcgccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggatccgccgataagggcgaattcataacttcgtataatgtatgctatacgaagttatggatctgtcgatcgacggatcgatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatggccgatcccatattggctgcacggatcctgaacggcagaggttacggcagtttgtctctcccccttccgggagccaccttcttctccaaccgtcccggtcgcgctctcggcgcttctgaggagagaactggctgagtgacgccctttatagattcgcccttgtgtcccgccccttcctttcccgccctcccttgcgctacggggccgcccgcaccggcctacacggagcgcgcgcggcggagttgttgacgctagggctccggctccctggttgggtgttctttctgacgcgacaggaggaggagaatgtcctggtcctgtcgtcctcctttcgggtttcccgtgcactcaaaccgaggacttacagaacggaggataaagttaggccatttttactcagcttcggagttcaggctcatttttcagctaaagtctctcattagtatccccccacacacatcgggaaaatggtttgtcctacgcatcggtaatgaaggcggggcccttcgggtcctccggagcgggttccgggggtggggggaaggagggagggacgggacgggcctcgttcatgaatattcagttcaccgctgaatatgcataaggcaggcaagatggcgcgtccaatcaattggaagtagccgttattagtggagaggccccaggacgttggggcaccgcctgtgctctagtagctttacggagccctggcgctcgatgttcaagcccaagctttcgcgagctcgaccgaacaaacgacccaacacccgtgcgttttattctgtctttttattgccgctcagctttacagtgacaatgacggctggcgactgaatattagtgcttacagacagcactacatattttccgtcgatgttgaaatcctttctcatatgtcaccataaatatcaaataattatagcaatcatttacgcgttaatggctaatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagttacccccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacgaacctggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcctgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacaccttcctcttcttcttgggcatggccgcaggaaagcagagccctgaagctcccatcaccggccaataagagccaagcctgcagtgtgacctcatagagcaatgtgccagccagcctgaccccaagggccctcaggcttgggcacactgtctctaggaccctgagagaaagacatacccatttctgcttagggccctgaggatgagcccaggggtggcttggcactgaagcaaaggacactggggctcagctggcagcaaagtgaccaggatgctgaggctttgacccagaagccagaggccagaggccaggacttctcttggtcccagtccaccctcactcagagctttaccaatgccctctggatagttgtcgggtaacggtggacgccactgattctctggccagcctaggacttcgccattccgctgattctgctcttccagccactggctgaccggttggaagtactccagcagtgccttggcatccagggcatctgagcctaccaggtccttcagtacctcctgccagggcctggagcagccagcctgcaacacctgcctgccaagcagagtgaccactgtgggcacaggggacacagggtggggcccacaacagcaccattgtccacttgtccctcactagtaaaagaactctagggttgcggggggtgggggaggtctctgtgaggctggtaagggatatttgcctggcccatggagatccataacttcgtataatgtatgctatacgaagttataagctttcgcgagctcgagatcctgcaggcgcgccgactggcgccggcgcgttctggcgacaggggagccaggggccgcggggaagcgaggactggcctgcgctgggctcgggagctctgtcgcgaggaggggcgcaggaccatggactgggggtggggcatggtggggattccagcatctgcgaacccaagcaatgggggcgcccacagagcagtggggagtgaggggatgttctctccgggacctgatcgagcgctgtctggctttaacctgagctggtccagtagacatcgttttatgaaaaggtaccgctgtgtgcattcctcactagaactcatccgacccccgacccccacctccgggaaaagattctaaaaacttctttccctgagagcgtggcctgacttgcagactcggcttgggcagcacttcggggggggagggggtgttatgggagggggacacattggggccttgctcctcttcctcctttcttggcgggtgggagactccgggtagccgcactgcagaagcaacagcccgaccgcgccctccagggtcgtccctggcccaaggccaggggccacaagttagttggaagccggcgttcggtatcagaagcgctgatggtcatatccaatctcaatatctgggtcaatccacaccctcttagaactgtggccgttcctccctgtctctcgttgatttgggagaatatggttttctaataaatctgtggatgttccttcttcaacagtatgagcaagtttatagacattcagagtagaaccacttgtggattggaataacccaaaactgccgatttcaggggcgggtgcattgtagttattattttaaaatagaaactaccccaccgactcatctttccttctctaagcacaaagtgatttggttattttggtacctgagaacgtaacagaattaaaaggcagttgctgtggaaacagtttgggttatttgggggttctgttggctttttaaaattttcttttttggatgtgtaaatttatcaatgatgaggtaagtgcgcaatgctaagctgtttgctcacgtgactgccagccccatcggagtctaagccggctttcctctattttggtttatttttgccacgtttaacacaaatggtaaactcctccacgtgcttcctgcgttccgtgcaagccgcctcggcgctgcctgcgttgcaaactgggctttgtagcgtctgccgtgtaacacccttcctctgatcgcaccgcccctcgcagagagtgtatcatctgttttatttttgtaaaaacaaagtgctaaataatatttattacttgtttggttgcaaaaacggaataaatgactgagtgttgagattttaaataaaatttaaagtaaagtcgggggatttccatccgtgtgccaccccgaaaaggggttcaggacgcgataccttgggaccggatttggggatcgttcccccagtttggcactagagacacacatgcattatctttcaaacatgttccgggcaaatcctccgggtctttttcacaacttgcttgtccttatttttattttctgacgcctaacccggaactgcctttctcttcagttgagtattgagctcctttataagcagacatttccttcccggagcatcggactttgggacttgcagggtgagggctgcgcctttggctgggggtctgggctctcaggagtcctctactgctcgatttttagatttttatttcctttctgctcagaggcggtctcccgtcaccaccttccccctgcgggtttccttggcttcagctgcggacctggattctgcggagccgtagcgttcccagcaaagcgcttggggagtgcttggtgcagaatctactaacccttccattccttttcagccatctccactaccctcccccagcggccacccccgccttgagctgcaaaggatcaggtgctccgcacctctggaggagcactggcagcgctttggcctctgtgctctttcctggggtcacctctgtctcctcttggccattgggttctcacaatccaaacccgcgatgcaaatttaggatgtggctgtgaagagagattctgggtggaaataaaaatactttggccttcctggtcaaggaccagggcagatcctgttgtagtctccgtgccccagggctggcctgagaatgagcccctgaaaagacagcgggtacgggcaccgtaagaacatcccctggtccagggtcctctctctgacaatatttttggtggccactggccaccctggaactgggggtgcagaagatttccccagtcagaaccccatttcttgagtcgcatagctgagcctggctcacacaggcaggcaccctttgcttagacttaaagactgctccgtcccctagcaagggacaggcacttcctgctcctccagcagggaatgtcggactgctggccagaacagcagtggcccagggattgggtgctggaggcctagtttttcaccgatgggcctggctttttgcaaaggctgggagggatttggagaggctgagcagctgggggctgaagacgggtggaaagcctcctgcccccaccaccccaacagcgccatgtgaatccaagaagaaggaagggcagggtgtagtcgtttttattctgaaatcccatttgaaatgaaacttgaaaagaattcaaaactgggtccagctgcagccacagacacactcagagggactccaggaggctggaacgtagaccagtgggcgctgagaacctggccggtgggggtaggggtcttgattgcagttttggctcttccacacccactgccaggcaggtgtactggtgcaggctctgagtgtgcttggtgtctgcatagaaggacggttgttgaaaggcaataaatcaagtctttccctccacccctgcacccaagctttcagtagcaaccagccaccagccaggccaggcaagaccagggcctctgaagaaggaggggctgtgtccagccaggctttgggccctcctccatgccagccgcctaaactgtgcacccagctggaggccttgaccacggtgggtgagactggagcagctctggacgtggaggaggaagacactggcacacagtgcacatcccctagaacaggtggctactcgccgagggtggccctggactggtgggggccaaggtagaggactcagccagtggctgggctttgatgtagggcaggagaagactgtgtgcaaccactttgactttggtgggctcttcattggcagtgggctcctcaccaagtagggaagggaaagaggtaactgtttccgggatctgctgcagtcttccctgccacactgcagtcccctctggggagcattcgttgggagattctcaacaggctggactgcagctcagcctcctccccagccccactgcgtggctctgcagcaccccagctgtcagcctccgagtttagaaactcaccatgccccaggtctttgagactccccaaccctccttaagaacctgggagctggccggggcgcagtggctcaagcctgtaatcccagcactgtgggaggccgaggtgggcagaccacctgaggttgggagttagagaccagcctgatcaaaatgcagaaactccgtctctattaaacatacaaaattagccagatgtggtggcacatgcctgtgatcccagctacttggcaggctgaggcaggagaagtgcttgtacccaggaggcggaggttgcaataagctgagatagtgccattgtactttgcactccagcctgagcaacaagagtgagactctgactaaaaaaaaagagaaaaaataaaaaaagaacttgggagctcagctgaccagaggttgaggggccagtgggcagaacccgagcctcctcctctggcctcagcaccccagggcattgtccctgggctcctcactctctcatatgggcaggagccacagagaaatgacactcccctggactcagtgttgaaggactaacccctaatgcccatggagttggaattctgcaccttggggacaggcagtgagcagacgagtgaagtgaacccacagaggtgtcacactcccagtgaggccaggagttttaatgtgaccgcaggaagacatgggtactaaagacacccgtggtgatttggggttctgacagccatcaccagggacctgcccttctcattcccagactgggatcagtgaggagcccctgggccagggcattgaattttaaagccccctagatgctcttatggataaaactaccccagtggtggaaaatgtctgtctcctattggggccgaatctacctagcaatctgtctggacttctattgagtattcaccttgatgcttcctaaaagttcatcaggaggtggcaaaggagtcccccttcacctgccatgtcattttaagtcactcttgcattacagattttgtgcctagtatcccccttccccacacaagtgcccccatagtaccagcaggggttatagggtctttgacaagggccccacaccaagagtgaccatttatttagtctccgcaaaactgtaagggaagccaggataaagccaggttattcacagcagtggttgctgtattcagtcccatcctgactagggagagggacgtgggacagcatgggtgagcatggccactccctggtttggaggcagccagcagctcatgggctatatcccagggctaagccccaactccctgtaagtgaggaggccattctaggcagggcagagatggggatcacccacagctacatccttactccgtatctctcctctagggccccagtgattgctactggctgtttgtctgtggaagaggttggaggtggaggactgcagcgaggaactgaagaggcaaagctgggaggagggagtcagggccgggctgaatctccagggaactcaggatattaataggtggaggctaggagaggaggcggatagcgttgactgaggtttagccggggtaaggcggggcacagagagctttaagagcctcagttcattccattctcactacaaccatttgaggagggtgctattattgtcccgtcttacaagtaaggaaactgagtcacagaaaggctaagtgacttgctcaggggacacagccagtaaatgaaggtggcaaggttgttgtaatccagggcccaccccaagaggccacctggcccgcactccagcagtccccagactctggctcagctgccgccccgggaatcctctgctgccaccccctgtcccccaccccggcccccttaggtggcgctcctagtgcttccccccatgcctggaccccatctgtccccgactcccccatctgggcggcgggcggggctcacaggctttagcaattagcgggcgcgggtgggcgcgcggcgcgggcggaggaagggccggagcaggtgagccgcggcggagcctcattagggcggcccgcacgccccgcgagggaccgtcgttagtgccaagacagctgccgctcctgccggcctagggccgcagccggaatcccgggccacatcccctgccccgttgtgccttccacacctcgggcagtcactaggaaaagggtcgccaactgaaaggcctgcaggaaccaggatgatacctgcgtcagtcccgcggctgctgcgagtgcgcgctctcctgccagggggacctcagaccctcctttacagcacaccgagggccctgcagacacgcgagcgggccttcagtttgcaaaccctgaaagcgggcgcggtccaccaggacgatctggcagggctctgggtgaggaggccgcgtctttatttggggtcctcgggcagccacgttgcagctctgggggaagactgcttaaggaacccgctctgaactgcgcgctggtgtcctctccggccctcgcttccccgaccccgcacaggctaacgggagacgcgcaggcccaccccaccggctggagaccccggcacggcccgcatccgccaggattgaagcagctggcttggacgcgcgcagttttcctttggcgacattgcagcgtcggtgcggccacaatccgtccactggttgtgggaacggttggaggtcccccaagaaggagacacgcagagctctccagaaccgcctacatgcgcatggggcccaaacagcctcccaaggagcacccaggtccatgcacccgagcccaaaatcacagacccgctacgggcttttgcacatcagctccaaacacctgagtccacgtgcacaggctctcgcacaggggactcacgcacctgagttcgcgctcacagatccacgcacaccggtgcttgcacacgcaagggcctagaactgcaaagcagcggcctctctggaccgcctccctccggccctcctgagccctactgagccctgctgagtcctggaggccctgtgacccggtgtccttggaccgcaagcatcctggtttaccatccctaccctggctgacctgagagctggggatcttttaagctttgtatgcctggcctcaccttttcctctgcctccctcccagaccagcccggctctcagacacctctgctttctaggcaaaagttctggagcccagagcaagggccacaggtgcagtgcagtccaaacaggaccacgagcaattctctctccttcatcctcagatttggccgcactcgagatatctagacccagctttcttgtacaaagtggttgattcgaggctgctaacaaatcgagtcgagcatcgagcagtgtggttttcaagaggaagcaaaaagcctctccacccaggcctggaatgtttccacccaatgtcgagcagtgtggttttgcaagaggaagcaaaaagcctctccacccaggcctggaatgtttccacccaatgtcgagcaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaacagcgtcaacagcgtgccgcagatcttggtggcgtgaaactcccgcacctcttcggccagcgccttgtagaagcgcgtatggcttcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgacccaagcttggctgcaggtcgagctcgcgaaagcttggcactggccgtcgttttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgt